(19) United States
(12) Patent Application Publication
(10) Pub. No.: US 2006/0155252 A1
(43) Pub. Date: Jul. 13, 2006

(54) Ostomy pouch washing device

(75) Inventors: Bart L. Walker, Payson, UT (US); Robert H. Oberg, Santaquin, UT (US)

Correspondence Address: MADSON & AUSTIN;GATEWAY TOWER WEST, SUITE 900, 15 WEST SOUTH TEMPLE, SALT LAKE CITY, UT, 84101, US

(73) Assignee: SW Technology, L.L.C.

(21) Appl. No.: 11/033,860

(22) Filed: Jan. 12, 2005

(51) Int. Cl. A61F 5/44

(52) U.S. Cl. 604/334

(57) ABSTRACT

A system attachable to a fluid supply and an ostomy pouch adapter wafer for washing an ostomy pouch releasably attachable to the ostomy pouch adapter wafer. The system includes a fluid delivery portion with a first end and a second end, the first end being attachable to the fluid supply. The system further includes a sealing plate having a support face and a fluid transport, the support face having an attachment surface for sealably receiving the ostomy pouch adapter wafer in releasable engagement. The fluid transport includes a fluid inlet attachable to the second end of the fluid delivery portion in a fluid-tight manner, a fluid outlet extending from the support face of the sealing plate to direct fluid into the ostomy pouch, and a one-way valve that permits outflow from the fluid outlet and prevents fluid outflow through the fluid inlet.

### BACKGROUND OF THE INVENTION

Some common surgical procedures involve the production of an “ostomy” in a patient to alleviate a symptom and/or improve the overall health of a patient. An ostomy is a surgically-created opening in a body wall that is connected to an internal structure such as a ureter or a small or large bowel to facilitate removal of waste from the body. Ostomies are most commonly produced to join portions of the intestines to the body wall and allow removal of digested food wastes. As a group, such procedures are referred to as “colostomies,” and generally involve the opening of the colon, or large intestine, to the exterior of the body. Such procedures may be temporary to allow portions of the colon to rest or heal following disease or surgery. Alternatively, colostomies may be permanent when a portion of the colon is lost.

By their nature, ostomies allow passive removal of wastes from the body by diverting them to a pouch affixed to the patient's skin as they flow through the intestines. Ostomy pouches are specially produced to capture and retain these materials as they exit the body in a discreet, odorless, and fluid-tight manner, and are regularly changed by the ostomate. These pouches come in a wide variety of shapes and configurations, but are most commonly attached to a patient by attaching a “wafer” to the patient that includes an adhesive surface to hold it in place, an orifice that is at least as large as the opening of the ostomy, and some type of adapter used to connect the wafer to the ostomy pouch. One common attachment system utilizes a temporary wafer that is placed on the body for a period of time such as a week. The wafer often has a ring of material that adheres directly to the patent's skin. The adhesive ring generally encircles a pouch adapter that mates with a corresponding structure on the pouch. These attachment structures often take the form of a protrusion that interlocks with a corresponding structure on the opposite portion of the device.

In the model in which a temporary wafer is adhered to a patient and ostomy pouches are periodically replaced as they fill with waste, the ostomy patient is faced not only with the emotional and physiological burdens of having an ostomy, but also with the financial burden of maintaining an adequate supply of adapter wafers and replaceable ostomy pouches. Such specialty medical products are relatively expensive, commonly costing from $200.00 to $300.00 per month to maintain a sufficient supply of pouches for normal usage.

Although in some cases, ostomy pouches are single-use products, patients often gravitate toward pouch models that are washable to allow re-use. Such reusable pouches often have an opening with an adapter that mates with a corresponding adapter wafer and a second sealable opening used to drain wastes and facilitate cleansing of the pouch for reuse. These reusable pouches reduce the financial burden on a patient by reducing the quantity of ostomy pouches needed to adequately care for the ostomy, but often result in an increased burden on the patient or caregiver by adding the unpleasant task of washing the used pouches. In addition, the use of reusable pouches is inconvenient when traveling, or when without facilities suitable for washing the pouch for an extended period of time.

Devices have been produced to simplify and somewhat automate the process of washing used ostomy pouches. These devices have historically been expensive and elaborate. In many cases, such devices have been so complex as to be difficult to use or so bulky that use away from home was very difficult. Such devices often feature spray nozzles that extend into the pouch from either the ostomy opening or the sealable opening to rinse the interior of the pouch with fluid and displace waste and debris. Often, no provision was made to seal the opening through which the spray nozzle entered. In the case of the ostomy opening, this was often the case because of the number of pouch and wafer systems currently available to ostomy patients rendered it difficult or impossible to provide a sealed opening. Washing using these devices thus often produced a clean pouch, but also often produced a spray emanating from one or another of the orifices that carried some of the waste matter with it. Such an unsanitary process has deterred many users from reusable pouches.

It would thus be a benefit in the art to provide a device that facilitates simple, sanitary washing of reusable ostomy pouches to allow their reuse by an ostomy patient. It would be a further benefit in the art to provide such a device that can be attached to the pouch in a sealed manner to prevent unintentional escape of the waste matter during the cleaning process. It would be another benefit to provide a device that facilitates cleaning of ostomy pouches that is compact and simple to use such that it is portable.

Such a device is provided herein.

### BRIEF SUMMARY OF THE INVENTION

Accordingly, the invention provides systems for washing an ostomy pouch. More specifically, the invention provides systems attachable to a fluid supply and to an ostomy pouch adapter wafer for washing an ostomy pouch that is releasably attached to the ostomy pouch adapter wafer. These systems generally include a fluid delivery portion and a sealing plate portion. The fluid delivery portion may broadly have a first end and a second end, the first end being attachable to a fluid supply. The second end of the fluid delivery portion is releasably and sealably attachable to a sealing plate having a support face for receiving an ostomy pouch adapter wafer, and a fluid transport. The support face of the sealing plate includes an attachment surface for sealably receiving an ostomy pouch adapter wafer in releasable engagement. The fluid transport includes a fluid inlet attachable to the second end of the fluid delivery portion in fluid-tight engagement, a fluid outlet extending from the support face of the sealing plate to direct fluid into the ostomy pouch, and a one-way valve that permits outflow from the fluid outlet and prevents fluid outflow through the fluid inlet.

In the systems of the invention, the fluid delivery portion of the system may include a flexible segment and an inflexible segment having the second end of the fluid delivery portion. In some instances, the inflexible segment of the fluid delivery portion may have a handle. Further, the inflexible segment of the fluid delivery portion may comprise a releasable attachment for securing the inflexible segment in fluid-tight engagement. The inflexible segment of the fluid delivery portion may further comprise a flow regulation valve. Additionally, the fluid inlet may be releasably attachable to the second end of the fluid delivery portion by using a quick-release mechanism.

The system may be coupled to a water source for use in a variety of ways. In some embodiments, the first end of the fluid delivery portion comprises a faucet adapter. In others, the first end of the fluid delivery portion comprises a tee for permanent integration into a fluid supply. Such a permanent attachment may additionally include a flow regulation valve. Each of the embodiments may include spray nozzle.

The invention further provides methods for cleaning an ostomy pouch using the pouch cleaning systems of the invention. The methods generally include the steps of releasably attaching an ostomy pouch adapter wafer to the sealing plate of the system described above. Following this, an ostomy pouch may be attached to the ostomy pouch adapter wafer in a sealed, releasable manner. The fluid delivery portion of the system may then be coupled to a supply of pressurized fluid to send fluid into the pouch. The ostomy pouch is then opened up at a pouch outlet such as a lower resealable opening to allow the contents of the pouch to escape. Following this, fluid is passed through the system into the ostomy pouch to carry out any waste present in the pouch and thus clean it.

The methods of the invention may be varied using variants of the system described above. The fluid delivery portion of the system may have a flexible segment and an inflexible segment, the inflexible segment being positioned intermediate to the flexible segment and the sealing plate. The inflexible segment of the fluid delivery portion may have a handle. The inflexible segment of the fluid delivery portion may have a releasable attachment for securing the inflexible segment to the flexible segment in fluid-tight engagement. The sealing plate of the system further may include a releasable attachment for securing the sealing plate to the fluid delivery portion in a fluid-tight manner. Further, the ostomy pouch adapter wafer may be attached to the support face of the sealing plate using an adhesive.

The invention further provides a plate releasably attachable to an ostomy pouch adapter wafer for sealing an ostomy pouch. Such a plate may include a sealing plate having a support face, and a fluid transport. The support face may have an attachment surface for sealably receiving the ostomy pouch adapter wafer in releasable engagement such that the wafer will remain attached thereto when an ostomy pouch is attached to the wafer. Beyond this, the wafer may be removable from the plate when forces are applied that are greater than those exerted by a full ostomy pouch to allow the wafer to be replaced.

The sealing plate may have a fluid inlet connectable to a fluid source and a fluid outlet extending from the support face of the sealing plate to direct a flow of fluid into the ostomy pouch. The fluid transport may further include a one-way flow valve that permits fluid outflow out of the fluid outlet and prevents fluid outflow from the fluid inlet. Additionally, the fluid outlet of the fluid transport may further comprise a spray nozzle. The one-way flow valve of the fluid transport may be positioned proximate the fluid outlet. Similarly, the fluid inlet of the fluid transport may comprise a quick-connect coupler for connection to a fluid source.

These and other features and advantages of the present invention will become more fully apparent from the following figures, description, and appended claims, or may be learned by the practice of the invention as set forth hereinafter.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a perspective view of an ostomy pouch washing device according to the present invention showing the device attached to a fluid delivery portion that allows it to be attached to a water source such as a toilet, sink, or other convenient pressurized source of water;

FIG. 2 is an exploded view showing the alignment of the sealing plate portion of the system of the invention with an ostomy pouch adapter wafer and an ostomy pouch for use;

FIG. 3 is a partially sectional side view of the sealing plate portion of the ostomy pouch washing system of the present invention shown mounted to an ostomy pouch for use;

FIG. 4 is an exploded side view of an embodiment of the system of the invention showing individual components which may be used to make up the pouch washing system of the invention;

FIG. 5 is an exploded side view of a second embodiment of the system of the invention showing individual components which may be used to assemble the second embodiment of the pouch washing system of the invention and alternative faucet adapters;

FIG. 6 is a frontal view of the sealing plate portion of the ostomy pouch washing device of the invention shown attached to an ostomy pouch adapter wafer prior to attachment of an ostomy pouch for washing.

### DETAILED DESCRIPTION OF THE INVENTION

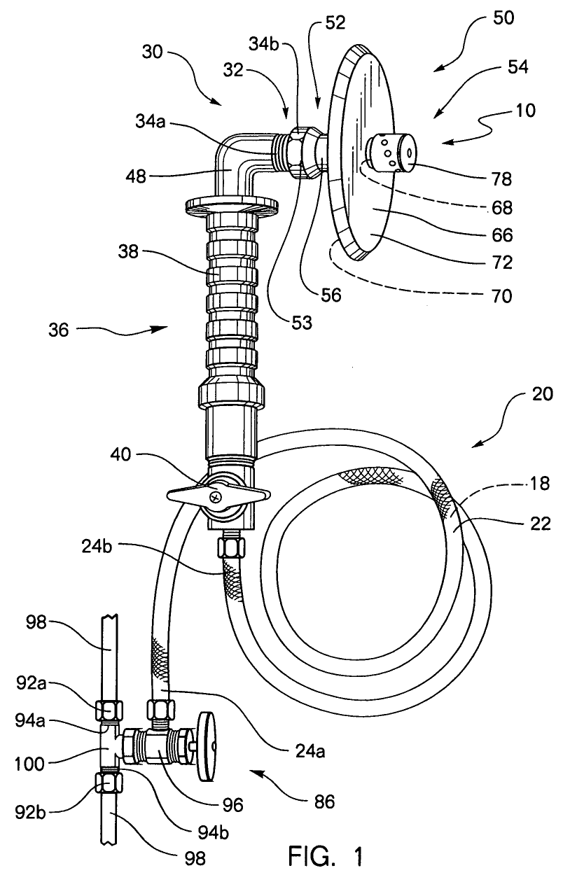

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the ostomy pouch washing device of the present invention, as represented in FIGS. 1 through 6, is not intended to limit the scope of the invention, as claimed, but is merely representative of presently preferred embodiments of the invention.

Referring first to FIG. 1, an ostomy pouch washing system 10 of the present invention is illustrated in a perspective view. In this view, the system 10 is assembled and shown in a configuration suitable for permanent or semi-permanent installation as a plumbed fixture. In some instances, the system 10, as shown, may be installed permanently into a fixture such as a toilet in a bathroom.

The pouch washing device 10 broadly includes a fluid delivery portion 20 for providing a flow of fluid through the system 10 and a sealing plate portion 50 for sealably attaching the system 10 to an ostomy pouch for cleaning. Each of these components will be discussed below in greater detail.

The fluid delivery portion 20 of the system 10 serves to deliver a flow of a fluid 18 from a source (not shown) to the sealing plate portion 50 to facilitate cleaning of an attached ostomy pouch. The fluid delivery portion 20 generally includes a flexible tube 22 that receives and conducts a pressurized fluid 18 from a fluid source into the system 10 and an inflexible portion 36 that also transmits the fluid 18. The flexible tube 22 is provided to allow the position, height, angle, etc., of the system 10 to be varied to facilitate its use. The length and size of the flexible tube portion 22 of the device may be varied by one of ordinary skill in the art to assure adequate length for use in specific installations, or may be standardized to facilitate economical system construction.

The flexible tube 22 includes a first end 24a and a second end 24b. As illustrated, the first end 24a is attached to a fluid source adapter 86. This adapter 86 is used to attach the system 10 to a water supply to provide fluid to the sealing plate portion 50 and allow cleaning of an attached ostomy pouch. This adapter 86 may take a wide variety of forms, including, but not limited to, an adapter that may be integrated with existing water supply lines such as those used to provide water to fixtures including, but not limited to, toilets, faucets, tubs, and showers, and adapters configured to receive fluid from an outlet of such a fixture. Providing such an adapter 86 allows the system to be mounted alternatively in a temporary, permanent, or even semi-permanent fashion to allow convenient use.

The fluid source adapter 86 has a valve 96 attached to a tee 100 inserted into a supply line 98. As briefly noted above, the supply line 98 may be a supply line attached to a fixture such as toilets, faucets, tubs, and showers. The tee 100 is shown to be held in place using paired compression nuts 92a, 92b and sleeves 94a, 94b. The valve 96 allows control over fluid flow channeled away from the fluid supply line 98. This may enable portions of the system 10 to be removed for cleaning, repair, or remote use without disruption of the existing plumbing. One of ordinary skill in the art would understand that the exact configuration of this permanently- or semi-permanently-installed fluid source adapter 86 may be widely varied within the art to allow fluid attachment of the system 10 to a wide variety of fluid supplies of different sizes, shapes, and designs.

In FIG. 1, the valve 96 is connected to the first end 24a of the tube 22 in a fluid-tight manner to assure proper transmission of fluid diverted from the tee 100. In the system 10 of FIG. 1, the second end 24b of the tube 22 includes a control valve 40. The control valve 40 may be used to allow control over fluid flow to the system 10 remotely from the fluid source adapter 86 and its included valve 96. Control valve 40 is capable of completely preventing fluid flow from the source into the inflexible portion 36 of the system 10. This may allow complete removal of the inflexible portion 36 and sealing plate 50 of the system 10.

The inflexible portion 36 of the fluid delivery portion 10 of the system 10 helps a user to control the system 10 during use. Control may be further improved by providing a handle 38 on the inflexible portion 36 to allow greater control. The design of the handle 38 may be varied widely, as known to those of skill in the art, to improve grip and accommodate right- and left-handed users. Such variations are within the scope of the present invention.

The inflexible portion 36 of the system of the invention may additionally include an angle segment 48 disposed between the handle 38 and a releasable attachment 30 used to attach the fluid delivery portion 20 of the system 10 to the sealing plate 50. When included, an angle segment 48 allows the sealing plate 50 to be oriented at an angle to the inflexible portion 36 of the system 10. This may also make it easier for a user to use the system 10. The angle segment 48 may be straight, or it may be designed to orient the sealing plate 50 at any of a wide variety of angles relative to the inflexible portion 36 of the system.

The inflexible portion 36 of the fluid delivery portion 20 of the system 10 terminates in a releasable attachment 30 used to couple the inflexible portion 36 to the sealing plate portion 50 of the system for use. This attachment 30 may take a wide variety of forms, including, but not limited to, threads or a quick-coupling assembly 32. The quick-coupling assembly 32 includes supply-side and outlet-side components 34a, 34b. In some instances, the supply-side component 34a of the quick-coupling assembly 32 is a male quick-coupler, and the outlet-side component 34b of the quick-coupling assembly 32 is a female quick-coupler. In others, this may be reversed within the scope of the invention. Further, alternative attachment means known to those of ordinary skill in the art may be utilized to releasably attach the sealing plate 50 to the fluid delivery portion 20 of the system 10.

As briefly discussed above, the system 10 further includes a sealing plate 50 for coupling the system 10 removably to an ostomy pouch in a sealed manner, and delivering the fluid supplied by the fluid delivery portion 20 to the pouch to clean it. The sealing plate portion 50 generally includes a fluid transport 52 and a plate 66. The fluid transport 52 couples releasably to the fluid delivery portion 20. The plate 66 receives an ostomy pouch adapter wafer (not shown) in a sealed, releasable manner such that an ostomy pouch may be attached to the plate 66 for cleaning.

The sealing plate portion 50 first includes a fluid inlet 53 for receiving a fluid 18 from the fluid delivery portion 20 of the system 10 when assembled. This inlet 53 may include the outlet-side component 34b of the releasable attachment 30 discussed above. This component 34b attaches the sealing plate portion 50 to the fluid delivery portion 20. The fluid transport 52 of the sealing plate portion 50 travels through an opening 68 of the plate 66 from a rear face 70 toward a support face 72 in a fluid-tight fashion. This fluid-tight seal may be provided using washers or other suitable seals (not shown) or sealing methods known in the art, or by producing the fluid transport 52 and the plate 66 as a single unitary component. The fluid transport 52 then extends outwardly from the support face 72 of the plate 66 and terminates in a fluid outlet 54. In use, when an ostomy pouch is coupled to an ostomy pouch adapter wafer releasably attached to the plate 66 of the sealing plate portion 50, the fluid outlet 54 extends into the pouch. The fluid outlet 54 may optionally include a spray nozzle 78 to facilitate cleaning of an attached pouch. Spray nozzle 78 may be modified to have a wide variety of spray patterns known to those of ordinary skill in the art to provide effective cleaning of a variety of ostomy pouches.

The fluid transport 52 further includes a one-way valve 56. The one-way valve 56 permits fluid to enter the inlet 53 of the fluid transport 52, travel through the fluid transport 52, and exit through the fluid outlet 54. The one-way valve 56 prevents fluid or other substances from flowing outwardly through the fluid inlet 53. To prevent accumulation of unwanted fluid or other substances within the fluid transport 52, the one-way valve 56 may be provided proximate the fluid outlet 54. The one-way valve 56 may, however, be provided at any point of the fluid transport, including the fluid inlet 53.

The sealing plate 50 may be used independently from the fluid delivery portion 20 of the system 10 to simply seal a filled ostomy pouch. This function may be very useful to an ostomate traveling or away from a water source compatible with the system 10, but in need of changing their ostomy pouch and reluctant to simply dispose of it. In such a situation, the ostomate may simply attach the sealing plate 50 to the pouch to seal its contents and save the pouch itself for cleaning at a more convenient time or place. At that time or place, the sealing plate 50 may be attached to the fluid delivery portion 20 of the device, and fluid supplied to the system to wash the pouch clean.

This function may also be accomplished by providing sealing plates that simply comprise a plate 66 with a support surface 72. Such blank sealing plates would not include a fluid transport 52, and would thus function only to seal ostomy pouches using an ostomy pouch adapter wafer 60 as an adapter. Pouches sealed with such blank sealing plates could be cleaned later by substituting the functional sealing plate 50 of FIG. 1 for the blank sealing plate and washing the pouch as discussed above.

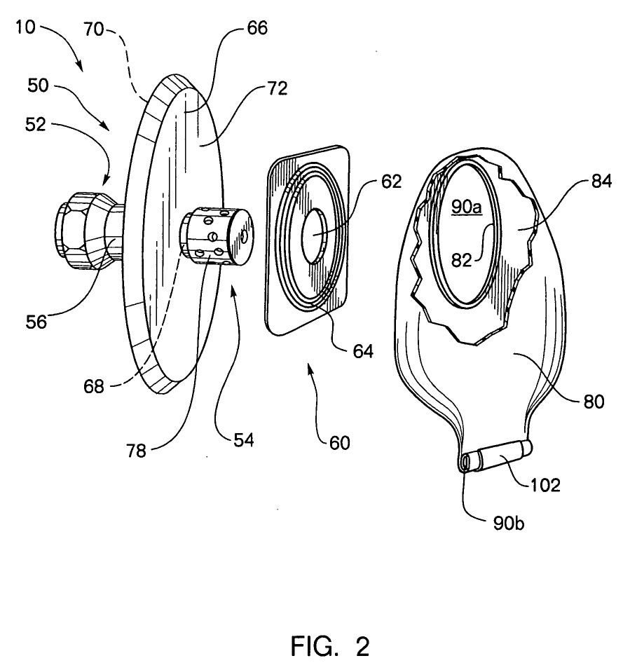

FIG. 2 provides an exploded side view of the sealing plate portion 50 of the ostomy pouch washing system 10 of the present invention, an ostomy pouch adapter wafer 60, and an ostomy pouch 80 shown properly aligned for sealable attachment to each other to allow cleaning of the pouch 80. A portion of the pouch 80 has been cut away to allow a view into its interior 84, and to show pouch opening 90a and the wafer attachment 82 used to attach the pouch 80 to the wafer 60. In this view, the property of the sealing plate portion 50 of receiving an ostomy pouch adapter wafer 60 in a sealed, releasable fashion may be attributed to the plate 66 having a support face 72 having a size, shape, and surface sufficient to receive the wafer 60. Such wafers 60 are commonly attached to the skin of ostomy patients using an adhesive. Such an adhesive may be placed on the skin-oriented face of the wafer 60 during its production to simplify the process of installing the wafer 60. Thus, for use, a backing is removed to expose the adhesive, and the wafer 60 is pressed against the patient's skin into place. Similarly, for use with the system of the present invention, any such backing is removed from the wafer 60 to expose the adhesive, and the wafer 60 is pressed against the support face 72 of the plate 66. The wafer 60 is oriented such that the wafer opening 62 is placed about the fluid outlet 54 and nozzle 78 of the fluid transport 52 extending from the support face 72 of the plate 66. This places the fluid outlet 54 within the interior 84 of the pouch 80 for use.

The wafer 60 includes a pouch attachment 64 to enable it to be sealably and releasably attached to the pouch 80 for use. In ordinary use, the wafer 60 depends from a patient's skin, holding to the patient with sufficient strength to support at least the weight of a full ostomy pouch 80. The pouch 80 may be separated from the wafer using a greater force. Further, the wafer 60 itself may be removed by the exertion of another force greater than that exerted by a full ostomy pouch 80.

Many companies are known to produce such two-piece wafer and pouch ostomy systems, each of which likely differs slightly in size and shape of the wafer 60, and the pouch attachment 64 on the wafer 60 and on the pouch 80. In some cases, the pouch attachment 64 may take the form of a ring having features that interact with those of a similarly-shaped feature of the pouch 80. Such an attachment 64 may be coaxial with an opening 62 of the wafer 60. This attachment 64 interacts with a corresponding pouch attachment feature 82 located on the pouch 80. Many types of such attachment mechanisms are known to those of ordinary skill in the art, and at least in part, the system 10 of the present invention is useful in that it accommodates a wide variety of pouch 80/wafer 60 systems produced by different companies. This provides flexibility to a patient in choosing and evaluating different products to meet their individual needs and preferences while retaining the ability to wash most such systems using the system 10 of the invention.

The ostomy pouch 80 has an ostomy opening 90a that includes a wafer attachment 82. When in use, the pouch 80 is placed such that the ostomy opening 90a receives waste from the patient's ostomy. The waste is stored within the interior 84 of the pouch 80. Reusable ostomy pouches 80 include a second sealable opening 90b, often at a lower extremity, used to drain accumulated waste from the pouch 80 and to facilitate cleaning of the pouch 80 for reuse. While in use, the second sealable opening 90b is closed in a fluid-tight manner to prevent escape of any of its contents. This closure may be made by rolling the pouch 80 against itself at the second sealable opening 90b and fastening the rolled portion in place using a device such as a clip 102, as illustrated. This allows the pouch 80 to be sealed repeatedly in a simple manner, thus allowing for multiple uses and easy cleaning.

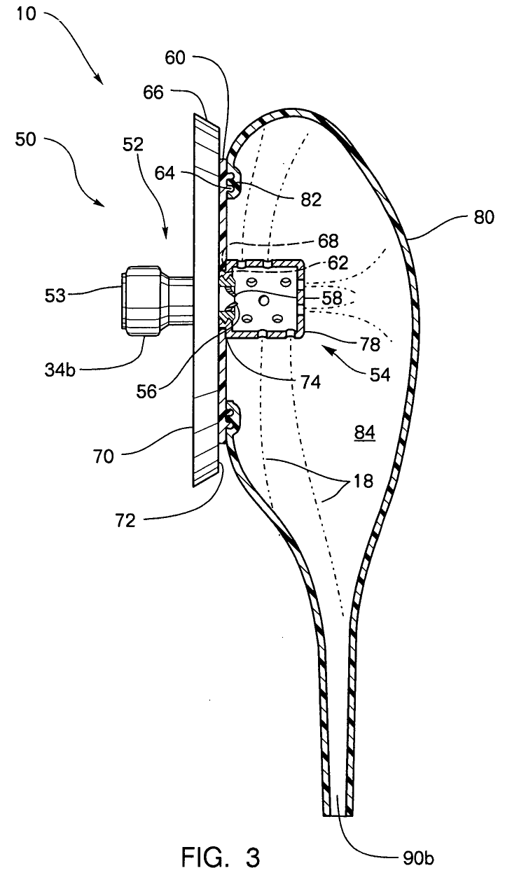

Referring next to FIG. 3, a partially sectional side view of the sealing plate portion 50 of the ostomy pouch washing system 10 of the present invention is shown. In FIG. 3, the sealing plate 50 is shown having an ostomy pouch adapter wafer 60 affixed thereto. More specifically, the wafer 60 discussed above is now shown to have been attached to the support surface 72 of the plate 66 of the sealing plate portion 50 of the system 10 of the invention. The wafer 60 has been placed such that the wafer opening 62 present in the wafer 60 encompasses the fluid outlet 54 of the fluid transport 52. As illustrated in FIG. 3, the fluid outlet 54 includes a spray nozzle 78 to produce a number of streams of fluid 18 directed outwardly from the nozzle 78 to help clean the pouch 80. In FIG. 3, the second pouch opening 90b of the pouch 80 is shown open to permit the fluid 18 to escape, carrying with it any waste accumulated in the pouch 80.

As discussed previously, the sealing plate portion 50 includes a fluid transport 52 and a plate 66. The fluid transport 52 extends from the outlet-side component 34b of the releasable attachment 30. In some instances, the outlet-side component 34b is a component of a quick coupling assembly 32. The fluid transport 52 then projects from the rear face 70 of the plate 66 through the plate 66 and out of the support face 72, terminating in the outlet 54, and in this case, the nozzle 78. As briefly discussed above, the sealing plate portion 50 of the system 10 of the invention may be assembled of a plurality of individual parts or produced as a single, unitary part. In those systems 10 in which the sealing plate portion 50 has been built of a plurality of parts, the plate 66 may include an opening 68 to accommodate the fluid transport 52. In systems 10 in which the sealing plate 50 has been produced as a unitary component, such a feature may not be necessary.

The fluid transport 52 includes a one-way valve 56 to prevent outflow of fluid 18 or material from the pouch 80 from the fluid inlet 53 of the fluid transport 52. The one-way valve 56 may be placed at any point along the transport 52. In some instances, the valve 56 may be placed near the fluid outlet 54 to prevent passage of significant amounts of fluid 18 and any other material from the pouch 80 into the fluid transport 52. As illustrated here, the one-way valve 56 includes gates 58 that open to allow passage of fluid out of the outlet 54, but close to prevent outflow from the inlet 53. Many one-way valves are known used in the art and would be understood by one of ordinary skill in the art to be useable within the scope of the present invention.

As noted above, the plate 66 has a support surface 72 to which the wafer 60 has been attached. This attachment is made such that the wafer 60 will be retained to the support surface 72 when subjected to loads at least as much as that exerted by a full ostomy pouch 80 on the wafer 60. Thus, the wafer 60 will be able to support an ostomy pouch 80 filled with water, fecal matter, etc., when attached thereto, without releasing from the plate 66. The wafer 60 is removable, however, when subjected to forces sufficiently greater than those exerted by a full pouch 80. This allows the wafer 60 to be removed and replaced as needed, such as when it becomes soiled, ceases to provide an adequate attachment or seal to an ostomy pouch 80, or when the user switches wafer 60/pouch 80 systems, thus necessitating the use of the new wafer 60 on the sealing plate portion 50 of the system 10.

The wafer 60 includes a wafer opening 62 to permit the fluid outlet 54 to extend from the sealing plate portion 50 of the system 10 into the interior 84 of the pouch 80. The opening 62 is surrounded by a wafer attachment 82 that interacts with the pouch attachment 64 of the wafer 60 to provide a sealed attachment of the wafer 60 to the pouch 80. As illustrated, this sealed attachment is provided by interaction of locking and sealing protrusions extending from both the wafer 60 and the pouch 80. Many suitable mechanisms for wafers 60 and pouches 80 are known in the art, and it is believed that many such mechanisms are suitable for use with the systems 10 of the invention.

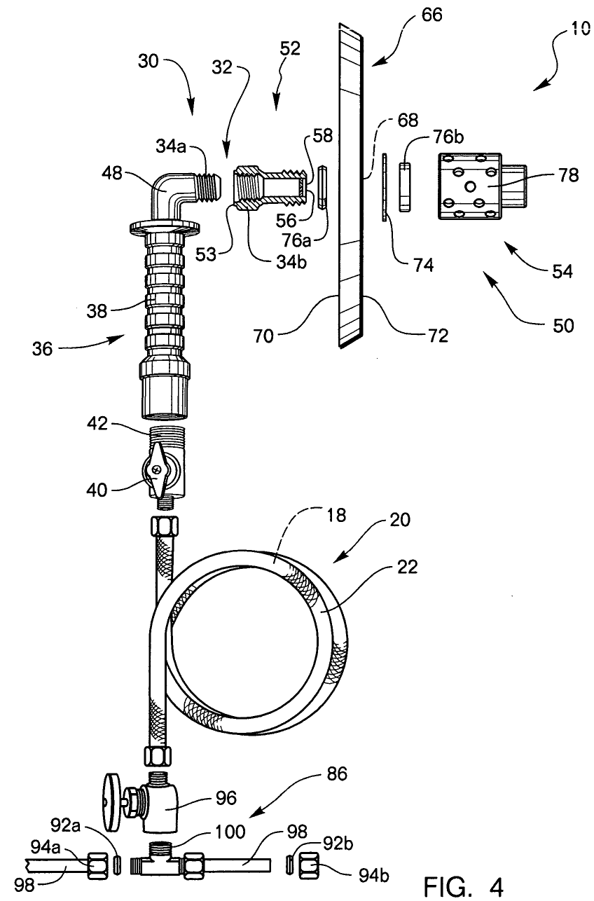

FIG. 4 is an exploded side perspective view of one embodiment of the ostomy pouch washing system 10 of the invention discussed with regard to FIGS. 1-3. The system 10 illustrated in FIG. 4 is shown exploded to illustrate the relationships between individual components which may be used to make up the pouch washing system 10 of the invention. It should be noted that although the system 10 illustrated in FIG. 4 is made up of many separate components, one of ordinary skill in the art could substitute components taking the place of more than one or less than one of the parts shown. Such modifications fall within the scope of the invention.

The system 10 illustrated in FIG. 4 includes a fluid delivery portion 20 and a sealing plate portion 50. The system 10 is shown to include a fluid source adapter 86 comprising, in this instance, a tee 100 coupled to a supply line 98 using paired compression nuts 92a, 92b and compression sleeves 94a, 94b. The tee 100 is, in turn, connected to a valve 96 that controls flow of fluid 18 from the tee 100. The valve 96 is continuous with a tube 22 extending away from the supply line 98. This tube 22 may be flexible to allow ease of movement of the system, as well as to assure ease of attachment of the system 10 to the valve 96. The length and dimensions of the tube 22 may be varied within the scope of the invention. The tube 22 is shown to connect to a control valve 40 sealably. The control valve 40 may connect to the inflexible portion 36 of the system 10 using a variety of means, including, but not limited to, quick-connect couplers, threads 42, or other means known to those of ordinary skill in the art.

The control valve 40 serves as an interface between the flexible tube portion 20 and an inflexible portion 36 of the system 10 that may generally include a handle 38 and the sealing plate portion 50. The handle 38 may be a separate component attached to the rigid angle segment 48. Alternatively, the handle 38 may be produced as a portion of a unitary angle component 48. As discussed above, the angle portion 48 is joinable to the sealable plate portion 50 of the system 10 by a releasable attachment 30, which is here shown to be a quick-connect coupling assembly 32. In some embodiments, the angle portion 48 terminates in the supply-side 34a of the quick-connect coupling assembly 32 in the form of a male quick-connect coupler.

The sealing plate 50 portion of the system 10 is here illustrated to include a fluid transport 52 having a fluid inlet 53, a fluid outlet 54, and a one-way valve 56. As illustrated herein, the one-way valve 56 is present in the outlet-side component 34b of the quick coupling assembly 32. The valve 56 is here illustrated to include valve gates 58 that prevent flow out of the fluid inlet 52. As noted previously, many suitable one-way valves 56 are known in the art and suitable for use in the present invention. The transport 52 is shown to be passed through a plate opening 68 for assembly, with a washer 74 and o-rings 76a, 76b being used to provide a fluid-tight seal with the plate 66. The sealing plate 66 then concludes with a spray nozzle 78 being attached to the outlet 54 of the transport 52.

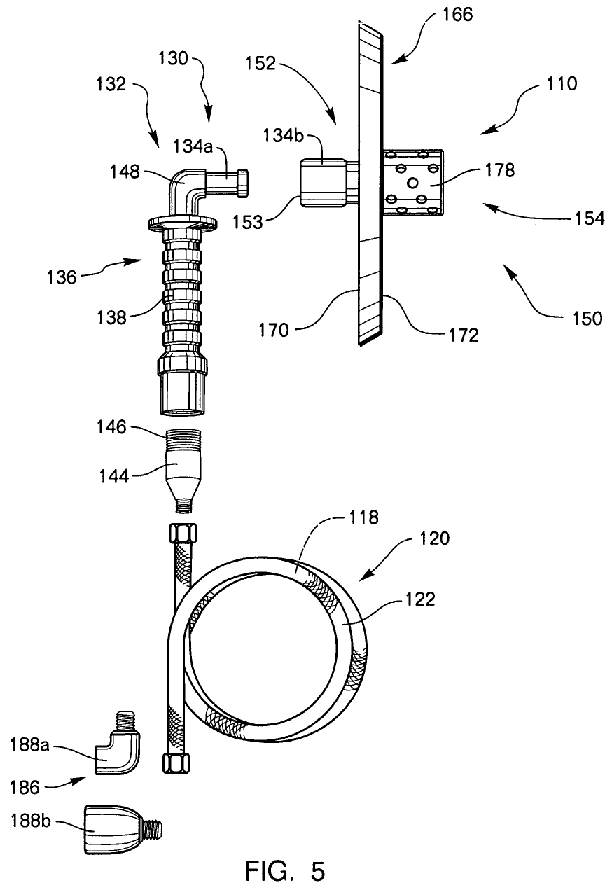

FIG. 5 provides an exploded side perspective view of a second embodiment of the ostomy pouch cleaning system 110 of the invention showing individual components oriented to show their orientation for proper assembly. In this embodiment of the system 110, variations are present in both the fluid delivery portion 120 of the system 110 and the sealing plate portion 150.

Referring first to the fluid delivery portion 120 of the system of FIG. 5, the fluid source adapter 186 is here shown to be configured for portable use. More specifically, in lieu of the permanent/semi-permanent tee 100 and valve 96 illustrated in FIGS. 1 and 4, the system 110 of FIG. 5 includes a flexible tube portion 122 for transmitting a fluid 118 attachable to an inflexible portion 136 of the system 110 in a fluid-tight manner by an adapter 144 having a connector such as threads 146. The tube 122 is then attachable to a fluid supply such as a faucet (not shown) via one of any number of a faucet adapters, of which two are illustrated as faucet adapters 188a, 188b. This allows the system 110 to be used portably by an ostomy patient by providing flexibility as to the fluid supply adapter 186.

The inflexible portion 136 of the system 110 includes, similar to that illustrated above, a handle 138 and an angle portion 148. The handle 138 may be a piece added about an extension of the angle portion 148 to connect to the fluid delivery portion 120, or it may alternatively be formed integrally. The angle portion 148 may angled as shown, at a wide degree of other angles as discussed above, or even straight. The angle portion 148 terminates in a releasable attachment 130 in the form of a quick coupling assembly 132 used to attach the inflexible portion 136 to the sealing plate portion 150. The quick coupling assembly 132 includes a supply side component 134a and an outlet side component 134b that includes an inlet 153 for the sealing plate portion 150. The quick coupling assembly 132 may include threads as illustrated in previous embodiments, or, as illustrated in FIG. 5, the supply side and outlet side components 134a, 134b may be male and female couplers capable of being rapidly attached in a sealed manner. Many such couplers are known to one of ordinary skill in the art and would be suitable for the practice of the present invention.

In addition to the above, the sealing plate 150 of the system 110 of FIG. 5 differs from that previously illustrated in that the fluid transport 152 and the plate 166 have been formed as a single unitary component. Thus, the fluid transport 152 extends continuously through the plate 166 in a sealed fashion without the use of washers, o-rings and other components. In some embodiments, the fluid transport portion 152 could be molded to include a quick-connect coupler 134b at its inlet 153 extending from the rear face 170 of the plate 166. Additionally, the one-way valve 156 may be produced within this unitary part, or it may be separately installed within the transport 152. The fluid transport 152 extends from the support face 172 of the plate 166, terminating in a fluid outlet 154. As shown, the outlet 154 includes a spray nozzle 178.

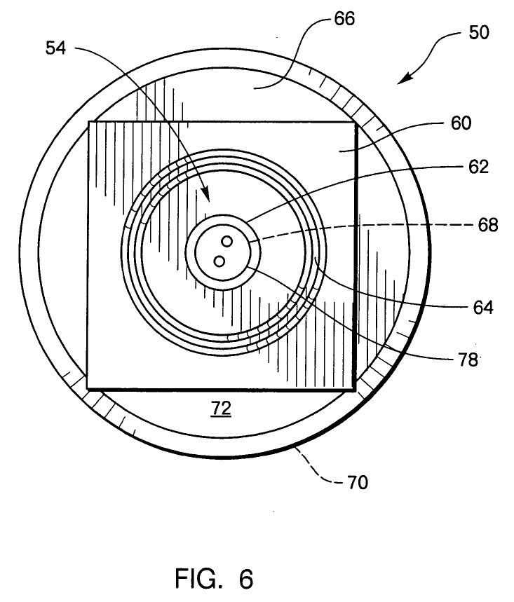

Referring last to FIG. 6, a frontal plan view of the sealing plate portion 50 of the ostomy pouch washing system 10 of the invention is shown with an ostomy pouch adapter wafer 60 attached to its support face 72 prior to attachment of an ostomy pouch for washing. In discussing this Figure, reference numbers have been provided that correspond to those given in FIGS. 1-4, but it is to be understood that these references correspond to similar components in FIG. 5. This view shows the interrelationship of the individual components that make up the sealing plate portion 50.

In FIG. 6, the plate 66 is shown to have received an ostomy pouch adapter wafer 60 on its support face 72. The plate 66 may also include an opening 68 to allow the fluid outlet 54 to extend through the plate 66 in systems 10 having sealing plate portions 150 composed of multiple components. The wafer 60 includes a pouch attachment 64 on its outer surface and a wafer opening 62, through which the spray nozzle 78 extends. The plate 66 of the sealing plate portion 50 of the system may be sized to accommodate a variety of wafers 60 to assure sufficient support surface 72 to allow sufficient attachment to retain a full ostomy pouch, as discussed above.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A system attachable to a fluid supply and an ostomy pouch adapter wafer for washing an ostomy pouch releasably attachable to the ostomy pouch adapter wafer, the system comprising:
a fluid delivery portion having a first end and a second end, the first end being attachable to the fluid supply;
a sealing plate having a support face and a fluid transport, the support face having an attachment surface for sealably receiving the ostomy pouch adapter wafer in releasable engagement, the fluid transport having a fluid inlet attachable to the second end of the fluid delivery portion in fluid-tight engagement, a fluid outlet extending from the support face of the sealing plate to direct fluid into the ostomy pouch, and a one-way valve that permits outflow from the fluid outlet and prevents fluid outflow through the fluid inlet.

2. The system of claim 1, wherein the fluid delivery portion of the system has a flexible segment and an inflexible segment having the second end of the fluid delivery portion.

3. The system of claim 2, wherein the inflexible segment of the fluid delivery portion comprises a handle.

4. The system of claim 2, wherein the inflexible segment of the fluid delivery portion comprises a releasable attachment for securing in fluid-tight engagement the inflexible segment.

5. The system of claim 2, wherein the inflexible segment of the fluid delivery portion further comprises a flow regulation valve.

6. The system of claim 1, wherein the fluid inlet is releasably attachable to the second end of the fluid delivery portion by using a quick-release mechanism.

7. The system of claim 1, wherein the first end of the fluid delivery portion further comprises a faucet adapter.

8. The system of claim 1, wherein the first end of the fluid delivery portion further comprises a tee for permanent integration into a fluid supply.

9. The system of claim 9, wherein the tee further includes a flow regulation valve.

10. The system of claim 1, wherein the fluid outlet further comprises a spray nozzle.

11. A method for cleaning an ostomy pouch using a pouch cleaning system, the system comprising a fluid delivery portion attachable to a fluid supply, a sealing plate having a support face and a fluid transport, the support face having an attachment surface for releasably engaging an ostomy pouch adapter wafer and the fluid transport having a fluid inlet attachable to the fluid delivery portion and a one-way valve permitting fluid outflow from a fluid outlet of the fluid transport and preventing fluid outflow through the fluid inlet, the method comprising:
releasably attaching the ostomy pouch adapter wafer to the sealing plate of the system;
attaching the ostomy pouch to the ostomy pouch adapter wafer in a sealed, releasable manner;
coupling the fluid delivery portion of the system to a supply of pressurized fluid;
opening a pouch outlet of the ostomy pouch; and
passing fluid through the system into the ostomy pouch to clean it.

12. The method of claim 1 1, wherein the fluid delivery portion of the system has a flexible segment and an inflexible segment, the inflexible segment being positioned intermediate to the flexible segment and the sealing plate.

13. The method of claim 12, wherein the inflexible segment of the fluid delivery portion comprises a handle.

14. The method of claim 13, wherein the inflexible segment of the fluid delivery portion comprises a releasable attachment for securing in fluid-tight engagement the inflexible segment to the flexible segment.

15. The method of claim 11, wherein the sealing plate of the system further comprises a releasable attachment for securing the sealing plate to the fluid delivery portion in a fluid-tight manner.

16. The method of claim 11, wherein the ostomy pouch adapter wafer is attached to the support face of the sealing plate using an adhesive.

17. A plate releasably attachable to an ostomy pouch adapter wafer for sealing an ostomy pouch comprising:
a sealing plate having a support face and a fluid transport, the support face having an attachment surface for sealably receiving the ostomy pouch adapter wafer in releasable engagement such that the wafer will remain attached thereto when an ostomy pouch is attached to the wafer, said wafer being removable from the plate when forces are applied that are greater than those exerted by a full ostomy pouch;
a fluid transport having a fluid inlet connectable to a fluid source, a fluid outlet extending from the support face of the sealing plate to direct a flow of fluid into the ostomy pouch, and a one-way flow valve that permits fluid outflow out of the fluid outlet and prevents fluid outflow from the fluid inlet.

18. The plate of claim 17, wherein the fluid outlet of the fluid transport further comprises a spray nozzle.

19. The plate of claim 17, wherein the one-way flow valve of the fluid transport is positioned proximate the fluid outlet.

20. The plate of claim 17, wherein the fluid inlet of the fluid transport comprises a quick-connect coupler for connection to a fluid source.
